(19) United States
(12) Patent Application Publication
(10) Pub. No.: US 2024/0415509 A1
(43) Pub. Date: Dec. 19, 2024

(54) ORTHOPEDIC STAPLE

(71) Applicant: Conventus Orthopaedics, Inc., Horsham, PA (US)

(72) Inventors: Darren DAHLIN, Plymouth, MN (US); Daniel SAVAGE, Plymouth, MN (US)

(21) Appl. No.: 18/742,556

(22) Filed: Jun. 13, 2024

Related U.S. Application Data
(60) Provisional application No. 63/507,967, filed on Jun. 13, 2023.

(51) Int. Cl. A61B 17/064; A61B 17/10

(52) U.S. Cl. CPC A61B 17/0642; A61B 17/10

(57) ABSTRACT

An orthopedic staple system and orthopedic staple are configured to facilitate bone healing through dynamic and adjustable compression. The system comprises the orthopedic staple having a bridge portion and one or more legs that anchor into bone structure. The bridge portion is configurable to include compression features, comprising a pre-compressed chevron, an “S” shaped deformable path, or other suitable geometries to enable active post-operative compression. The staple can be configured to accommodate a single screw or multiple screws, which interact with the bridge to apply targeted compressive forces. Screws can be seated in either fixed or translatable pockets within the bridge, facilitating variable angle adjustments and bone remodeling. This system is optimized for minimally invasive surgical applications, promoting healing and integration, and enhancing overall patient outcomes.

### CROSS-REFERENCE TO RELATED APPLICATION

This application claims the benefit of U.S. Provisional Application No. 63/507,967, filed Jun. 13, 2023, the entirety of which is incorporated herein by reference.

### BACKGROUND

Conventional staple systems used in orthopedic surgeries have been designed to provide mechanical compression across a fixation site for proper healing of bone fractures. These systems typically include a staple with a rigid bridge that connects two penetrating legs, anchoring the staple into the bone. However, a significant limitation of these existing designs is the inability to apply compressive force at the top of the staple, specifically the bridge area. This region remains substantially rigid and does not participate in the dynamic movement needed for effective compression of the bone segments. As a result, the area of the bone immediately adjacent to the bridge of the staple, particularly the near cortex, does not benefit from the necessary compressive forces that facilitate optimal healing and bone remodeling. The static nature of traditional staple bridges thus represents a critical shortfall in the ability of current systems to address the comprehensive needs of bone healing, especially in complex or particularly delicate bone structures.

### BRIEF SUMMARY OF THE INVENTION

According to some embodiments, an orthopedic staple system is disclosed that is designed to overcome the limitations of conventional staple systems by providing active, constant compression not only across the fixation site but also at the bridge of the staple, thus encompassing the entire joint. This is achieved through several innovative principles and features integrated into the staple design.

According to some embodiments, a single-sided staple is disclosed that is paired with a screw positioned on the opposite side of the staple leg(s). The staple can accommodate the screw in either a simple hole or a slot, the latter allowing for variable positioning of the screw to accommodate bone compression and remodeling dynamically. The use of slots enables the screw head to migrate within the staple, facilitating adaptive compression as the fracture site heals and the bone structure remodels. The screw is configured to provide active compression, which effectively enhances the interaction between the leg side of the staple and the hole or slot side, ensuring comprehensive application of compressive forces.

According to some embodiments, the staple is configured to incorporate a compression element within the bridge of the staple. This element can be pre-compressed in a chevron shape, or alternatively, “S” shapes or multiple chevrons can be utilized, depending on the specific surgical requirements and desired compression characteristics. A compression portion, e.g., the chevron of the bridge, can be configured to be compressed (e.g., along the transverse axis) prior to insertion and then released once the staple is fixed in place, causing the bridge to shorten and thus add to the overall compression exerted by the system. The compression portion can be expanded prior to insertion and then released to enhance compression during a procedure. as the chevron or “S” shapes in combination with an adjustable screw mechanism in the staple system presents an improvement over conventional orthopedic staple systems.

The staple is configured to resolve the unaddressed need for active compression constructs that can deliver consistent and adaptive compression across the entirety of the joint, including critical areas adjacent to the bridge of the staple. This system significantly advances the field of orthopedic fixation by offering a more effective, adaptable, and responsive approach to bone healing.

### BRIEF DESCRIPTION OF THE DRAWINGS/FIGURES

FIG. 1A is an illustration of an example orthopedic staple system, according to some embodiments.

FIG. 1B is an illustration of an example “S” shaped orthopedic staple system, according to some embodiments.

FIG. 1C is an illustration of an example orthopedic staple system having a double chevron, according to some embodiments.

FIGS. 2A to 2C are illustrations of an example orthopedic staple system, according to some embodiments.

FIGS. 3A and 3B are illustrations of an example orthopedic staple system, according to some embodiments.

FIG. 4 is an illustration of an example orthopedic staple system, according to some embodiments.

FIG. 5 is an illustration of a surgical application of an orthopedic staple system, according to some embodiments.

### DETAILED DESCRIPTION OF THE DRAWINGS

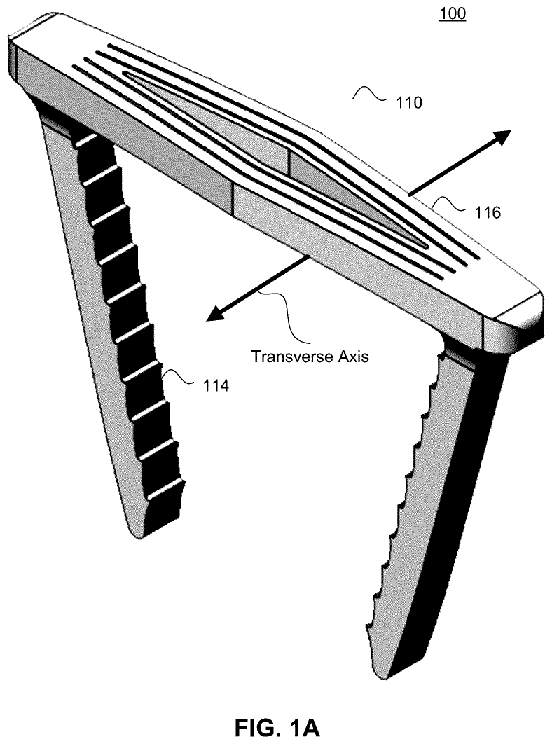

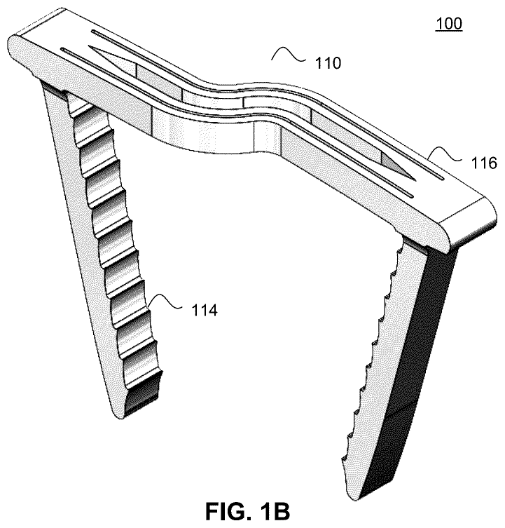

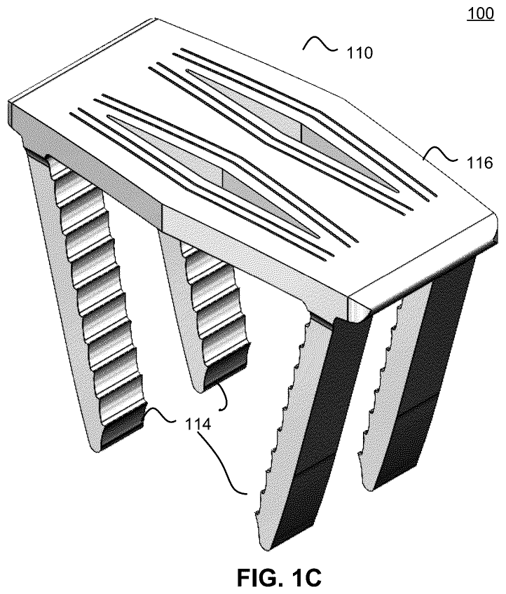

FIG. 1 illustrates an embodiment of an advanced orthopedic staple system 100, designed for effective and adjustable compression across a bone healing site, particularly leveraging the dynamic properties of one or more elastically deformable polygons. For example, some embodiments can include one or more elastically deformable polygons in the form of a chevron-configured bridge. The term ‘chevron,’ as used herein, can refer to a V-shaped configuration characterized by two arms extending at an angle from a common junction, which may be a point, a curved segment, or a linear segment, such as part of a circle or trapezoid, resembling an inverted ‘V.’ In some non-limiting examples, this chevron shape can be employed in the bridge element of the orthopedic staple to enhance the dynamic application of compression across the fracture site. The arms of the chevron can be configured to exert compressive forces when deployed, contributing to the reduction of the bridge's length and effectively increasing the pressure exerted by the staple on the healing bone. Additionally, the chevron shape can encompass a double (or plural) V shape, or double (or plural) chevron as a configuration where two or more V-shaped patterns are aligned along their respective centers, creating a series of four or more arms that can provide a balanced and distributed compressive force, thereby accommodating more complex or larger bone structures. This configuration can provide an improvement for maintaining stability and uniform compression across wider or more irregular fracture sites. This embodiment of the staple's design (shown here without the screws, which can be optionally included), emphasizes the mechanical and functional aspects that enhance the compression delivered directly to the bone during the healing process. The innovative design integrates a combination of material science and biomechanical engineering to optimize healing dynamics by distributing uniform compression across the affected area.

In some embodiments, orthopedic staple system 100 can include orthopedic staple 110. Constructed using biocompatible, robust materials suitable for orthopedic implants such as titanium or medical-grade stainless steel, to name just two non-limiting examples, orthopedic staple 110 exhibits a U-shaped configuration with multiple legs 114. Each leg 114 can be designed to penetrate and securely anchor into the bone, providing stability and support. The design of these legs, depicted as having a straightforward, elongated form in the illustrated embodiment, extends from each end of a central bridge element 116, which plays a pivotal role in the compression mechanism. This structural arrangement can ensure that the staple maintains its positional integrity while applying necessary forces to the bone healing site.

Orthopedic staple 110 comprises bridge element 116, which can employ a chevron configuration. Bridge element 116 may also incorporate optional slots to allow for adjustable positioning of one or more screws (not shown). These slots can be positioned to align with the arms of the chevron and may be partial or through-going, depending on the requirement for screw mobility and the specific application of the staple. Prior to insertion, the chevron can be mechanically compressed, preloading the bridge 116 with potential energy. This pre-compression can be beneficial as it can be released once the staple 110 is in place exerting additional compressive force across the fracture site. The potential energy stored within the bridge element 116 can be calibrated to enhance the natural healing process by applying consistent, targeted pressure to promote bone growth and alignment.

The chevron shape of the bridge 116 allows it to shorten when the compression is released, pulling the legs of the staple 110 closer together and enhancing the overall compression at the bone healing site. This dynamic adjustment can be beneficial for maintaining constant, optimal pressure on the healing bone, accommodating slight movements and natural bone growth during the recovery process. The ability of the bridge 116 to dynamically adjust its form and apply varying degrees of pressure can enable a responsive and adaptive solution to bone healing.

As shown, according to a non-limiting embodiment, orthopedic staple 110 can be configured with a chevron shape. System 100 can additionally or alternatively accommodate other shapes, such as “S” shapes (e.g., as shown in FIG. 1B) or different combinations of chevrons (e.g., double chevron as shown in FIG. 1C). Each configuration has specific attributes regarding how compression can be applied and adjusted post-insertion, allowing for tailored treatment approaches based on the specific medical requirements and the nature of the bone injury. These variations are designed to meet diverse clinical needs, providing surgeons with multiple options to optimize patient outcomes.

The interaction between the pre-compressed bridge 116 and the legs 114 of the staple can be engineered to provide a balanced, uniform compression directly to the healing site. Upon insertion and activation (release of the pre-compressed bridge 116), the staple 100 not only stabilizes the bone but also actively promotes healing by enhancing the mechanical environment at the fracture site. This compression can be beneficial for achieving rapid and effective bone regeneration.

Staple system 100 can be particularly advantageous in surgeries where precise and adaptable compression is necessary to support the healing of complex fractures. The ability of the bridge 116 to impart additional compression after insertion makes this system highly effective in maintaining the required mechanical stability and promoting optimal bone healing. The option to preload the bridge 116 with different levels of compression and the ability to use various bridge 116 shapes offers significant flexibility in surgical applications. Enhanced compression across the staple 110, facilitated by the dynamic bridge element 116, ensures better adaptation to the bone healing process, potentially reducing healing time and improving outcomes. Despite its advanced functionality, the system 100 remains straightforward to install, with the pre-compression of the bridge 116 simplifying the application of effective compression, thus streamlining the surgical procedure and reducing operative time.

Staple system 100 can be fabricated with materials selected to provide robustness and biocompatibility suitable for a bone stapling application. In a non-limiting example, staple system 100 can be fabricated using materials such as ultra-high molecular weight polyethylene (UHMWPE) and/or polyether ether ketone (PEEK), which may provide superior mechanical and biological properties. For example, UHMWPE provides exceptional durability, high impact resistance, and minimal wear properties, and may be preferred in orthopedic applications involving high loads and repetitive movements. PEEK provides excellent stiffness, radiolucency, and compatibility with medical imaging tools, in addition to providing significant mechanical properties that make it suitable for dynamic applications within the human body. The use of these materials can significantly enhance the performance and acceptance of the staple, ensuring long-term reliability and functionality. Fabrication of staple system 100 can incorporate techniques such as forging and laser sintering. Forging can increase the material density and alignment of the molecular structure, enhancing the implant's mechanical strength and fatigue resistance. In some embodiments, laser sintering may be performed to create complex geometries that traditional methods cannot, allowing for precise control over the microarchitecture of the staple for optimal integration and performance in the body. These manufacturing processes can ensure that the staple can withstand the stresses it will encounter during surgery and post-surgery, thus maintaining its integrity and functionality throughout the healing process.

The design of orthopedic staple system 100 enables efficient distribution of load across the healing site. The staple legs 114 and the bridge 116 can be configured to ensure that forces exerted during and after implantation are not concentrated in a single area but are spread across the staple and into the bone uniformly. This load distribution can reduce peak stress points at the bone-implant interface, which can lead to complications such as local bone degradation or implant loosening over time. The mechanical interaction of the staple legs with the bone can be configured to support the natural healing process, accommodating slight bone movements and adjustments without compromising the stability or integrity of the fixation. This approach enhances the healing environment and ensures that the implant can handle the mechanical demands of the body during the recovery period.

Staple system 100 can provide adaptable compression mechanisms that allow for post-operative adjustability, enabling optimal compression as the healing process progresses. The design includes adjustable features that can be externally modified to tailor the compression to the specific needs of the healing bone. This adaptability is achieved through the incorporation of mechanical elements within the staple 110 that can be fine-tuned after insertion. For example, the bridge 116 of the staple 110 may include adjustable tensioning mechanisms that can be accessed and adjusted through minimally invasive procedures. This capability ensures that the applied compression can be dynamically controlled, adapting to changes in bone density, swelling, and other physiological factors that might occur post-surgery. Such adjustability not only enhances the efficacy of the treatment but also reduces the need for additional surgeries to re-adjust the staple, thereby improving patient outcomes and reducing overall treatment costs. The ability to fine-tune the compression post-insertion is a significant advancement over traditional static compression systems, offering a more personalized and responsive approach to bone healing.

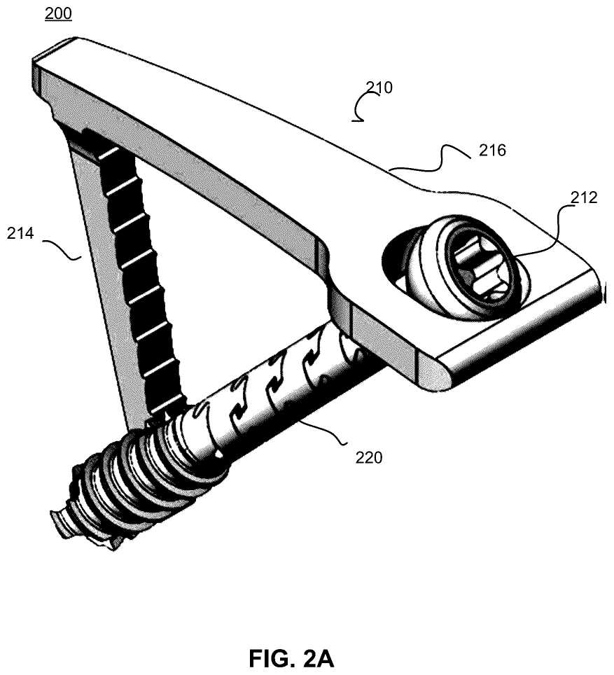

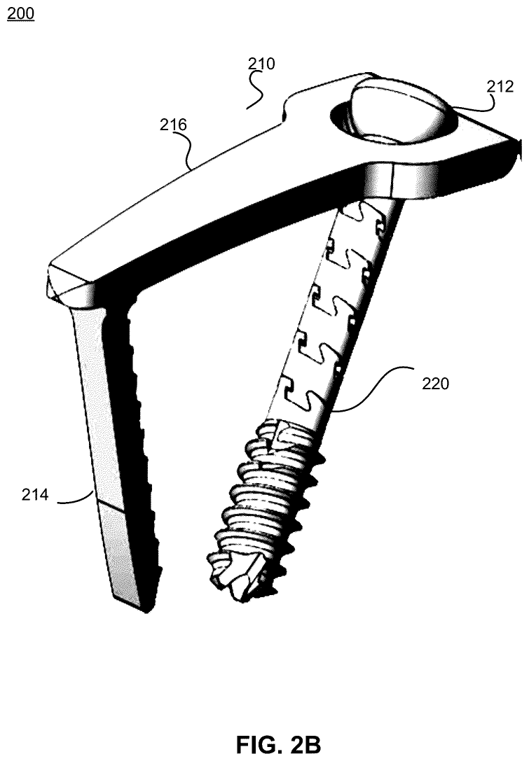

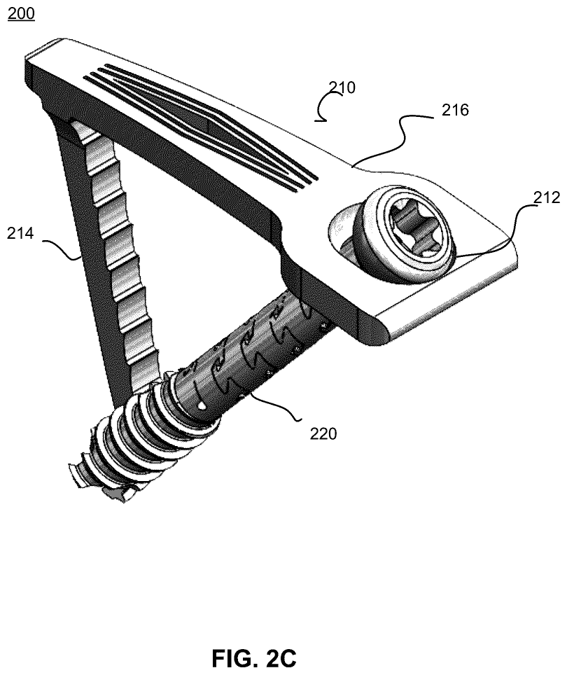

FIGS. 2A to 2C illustrate a detailed configuration of an orthopedic staple system 200, which can be part of a broader medical device designed to aid in bone healing by applying targeted compression through a single-leg staple and a fixed compression screw. FIGS. 2A to 2C depict staple system 200 that can include staple 210 including a bridge 216 coupled with a single leg 214 of the staple, designed for direct insertion into bone structure. The leg 214 can be configured to penetrate the bone with precision and remain securely in place, providing stable support at the site of implantation.

The leg 214 can alternatively be part of a larger U-shaped staple configuration that includes an additional leg coupled with connecting bridge 216. The upper part of this additional leg 214 can be configured to interface with the compression screw 220, where it plays an additional role in the compression mechanism of the staple system 200. The leg 214 extends vertically and features a straight, robust design optimized for ease of insertion and maximal contact with bone structure, ensuring a strong anchorage.

Positioned next to the staple leg 214 is the compression screw 220. This screw can be specifically designed to be inserted through a fixed hole 212 located at the bridge adjacent to the proximal end of the leg 214. The hole 212 can be precisely engineered to accommodate the screw 220, ensuring that once inserted, the screw 220 remains firmly in place without significant lateral or longitudinal movement. This fixed positioning of the screw 220 can be beneficial for the screw's function, as it allows the screw 220 to apply a constant and controlled compressive force directly to the leg 214, and consequently to the bone.

The fixed hole 212 contrasts with other designs that may use a slot to allow for adjustable positioning of the screw 220. The choice of a fixed hole ensures that the compressive force delivered by the screw is stable and unvarying, providing a consistent therapeutic benefit that can facilitate the healing process. This setup can be particularly beneficial in clinical situations where a precise amount of compression needs to be maintained without the risk of post-operative adjustments.

Moreover, the interaction between the leg 214 and the screw 220 in this configuration can be designed to maximize the compression effect by leveraging the mechanical advantage of the screw's placement relative to the leg's insertion point. As the screw 220 is tightened, it exerts downward pressure on the leg 214, which in turn transmits this force into the bone, enhancing the compression at the fracture or osteotomy site. This mechanism can promote optimal bone healing, as it ensures that the bone segments are held firmly in place, reducing movement at the fracture site and allowing the natural healing processes to proceed with minimal disturbance.

The design and functionality of the system 200 depicted in FIGS. 2A to 2C can enhance bone healing, by improving the precision and stability of the compression applied. This system 200 can be ideally suited for applications where the surgical site is sensitive to variations in compression or where a very controlled healing environment is required. The specific attributes of the staple leg 214 and the fixed compression screw 220, as illustrated in FIGS. 2A and 2B, are indicative of a sophisticated understanding of biomechanical principles, tailored to meet the rigorous demands of orthopedic surgery and bone healing therapies.

FIG. 2B illustrates another perspective of the orthopedic staple system 200 to deliver targeted mechanical pressure for effective bone healing. As noted above, staple 210 includes a single leg 214 that can be part of a broader system potentially featuring a bridge with integrative compression features. The screw 220 can be seated in a variable angle pocket 212, a hole without translation, ensuring that screw 220 remains fixed relative to the staple 210 while allowing for angular adjustments to accommodate the anatomy and positioning required during surgery. This configuration can enable, for example, procedures involving small bones or in areas with limited space where precise placement is important.

FIGS. 2A and 2B depict staple 210 without a chevron-shaped bridge. FIG. 2C illustrates alternative staple 210 that includes a chevron that can be compressed to preload the bridge with potential energy. As in the embodiment of FIGS. 1A to 1C, potential energy stored within the chevron-shaped bridge element 216 can be calibrated to enhance the natural healing process by applying consistent, targeted pressure to promote bone growth and alignment.

The surgical application of this configuration could typically involve scenarios like wrist, foot, and/or ankle surgeries where small, precise compressions are necessary to ensure proper bone alignment and healing. The fixed nature of screw 220 in this embodiment ensures that once the desired compression is achieved during surgery, it remains consistent, providing ongoing support as the bone heals.

Alternatively, pocket 212 can be configured to permit the translation of screw 220. This allows one or more screws to move slightly within its slot, accommodating natural bone movements and changes during the healing process, thereby maintaining optimal compression throughout.

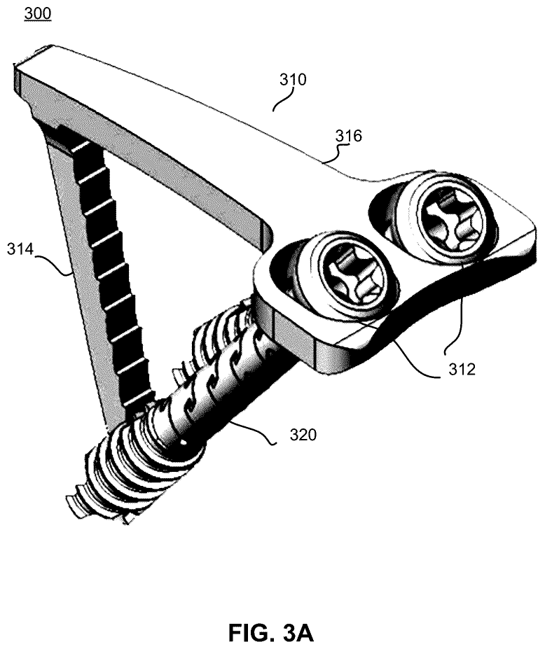

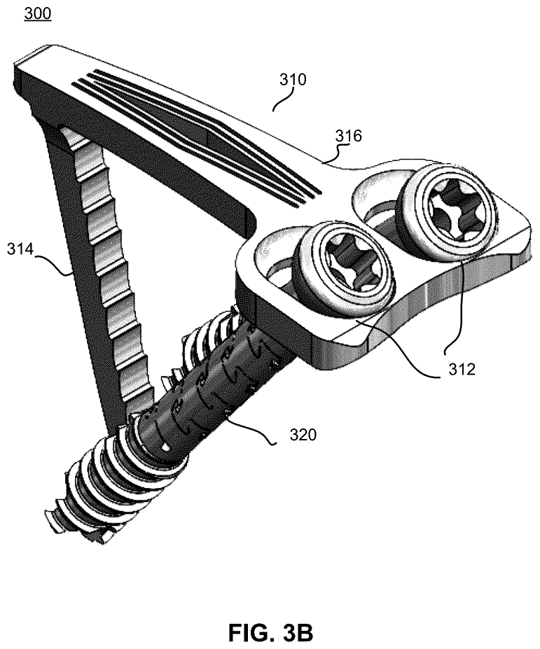

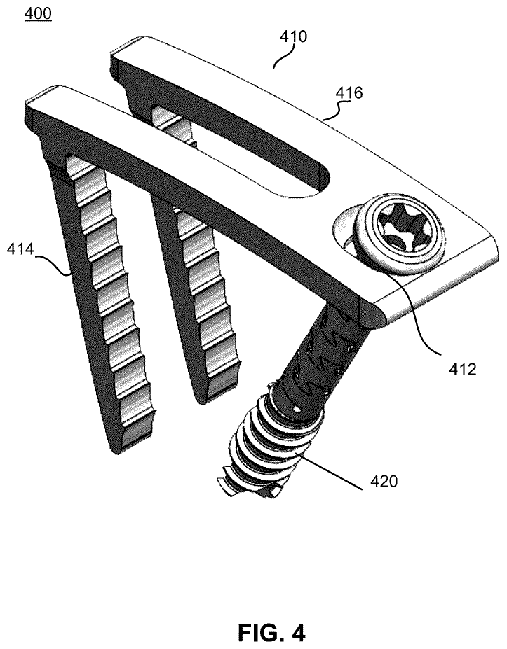

FIGS. 3A and 3B illustrate another embodiment within the orthopedic staple system, indicated generally at 300. The design incorporates a plural-pocket (or, alternatively, a slot) configuration within the staple's bridge to accommodate two or more compression screws. This configuration can be part of a sophisticated system designed to provide enhanced and adjustable compression across a bone healing site through the use of multiple screws. FIG. 3A depicts staple 310 without a chevron-shaped bridge. FIG. 3B illustrates alternative staple 310 that includes a chevron that can be compressed to preload the bridge with potential energy. As in above embodiments, potential energy stored within the bridge element can be calibrated to enhance the natural healing process by applying consistent, targeted pressure to promote bone growth and alignment.

Staple 310 depicted in FIGS. 3A and 3B includes a bridge that extends horizontally from the top of the single leg 314, similar to the embodiment of system 200 depicted in FIGS. 2A and 2B. Leg 314 extends vertically to ensure deep penetration into the bone for stable anchorage. The leg's robust and straightforward design can facilitate insertion into the bone, maximizing contact and stability. However, unlike the configuration in FIGS. 2A and 2B, system 300 can include two or more pockets or slots 312 positioned to allow for the insertion of two separate compression screws.

The plural slots 312 are each configured to receive a compression screw 320 and can be placed along the bridge 316 to maximize the distribution of compressive force when the screws 320 are inserted and tightened. Each slot 312 can be engineered to ensure that the screws 320 can be adjusted within a certain range, allowing for fine-tuning of the compression based on the specific needs of the bone healing process. This adjustability can enable varying degrees of compression that may be necessary to accommodate different stages of healing or variations in bone density.

The inclusion of plural screws 320 enhances the system's ability to apply uniform compression across the site of implantation. As each screw 320 is tightened, it independently exerts pressure on the bridge element 316, which in turn pulls the leg 314 inward towards the center of the staple 310. This action can facilitate compression of the bone fragments together, enhancing the healing process by maintaining bone alignment and stability. The plural screw system 300 also offers redundancy, ensuring that if one screw's compression is insufficient or loses its grip, the other can continue to provide necessary compression, thereby maintaining the integrity of the staple's function.

This plural-pocket configuration 300 can be particularly advantageous in complex surgical applications where precise control over the compression applied is important. The ability to independently adjust each screw 320 allows surgeons to tailor the force applied to each side of the staple 310, accommodating asymmetrical bone structures or uneven fracture lines. Furthermore, the ability of each screw 320 to slide within its respective slot 312 provides a dynamic compression mechanism that can adapt to changes in bone structure or swelling during the healing process, ensuring continuous and effective compression.

The design and functionality of the staple system 300 depicted in FIG. 3 exemplify an advanced approach to bone healing, where precision, adaptability, and reliability are paramount. This system can be ideally suited for challenging orthopedic conditions where meticulous control over the mechanical environment is necessary to ensure successful healing outcomes. The specific attributes of the plural-pocket 312 configuration, combined with the robust design of the leg 314 and the strategic placement of the screws 320, highlight a comprehensive understanding of biomechanical principles tailored to enhance therapeutic efficacy in bone healing therapies.

This configuration utilizing multiple screws 320 can include a plate or staple that spans a larger area, with slots 312 that permit the translation of screws 320. This feature allows each screw 320 to move slightly within its slot 312, accommodating natural bone movements and changes during the healing process, thereby maintaining optimal compression throughout.

The bridge 316 of the staple 310 in this illustration incorporates features that provide active compression post-operatively. This can be achieved through the integration of elastically deformable polygons or a deformable bridge path, such as an “S” shape, or any other bridge compression configurations disclosed herein, which can actively contribute to the compression force as the bone undergoes natural healing phases. Such dynamic features are particularly advantageous in surgeries involving larger bones or segments where the healing process may be prolonged, and the bone undergoes significant remodeling.

For example, in a clinical scenario involving the repair of a large fracture in the femur, the ability of the screws 320 to translate within their slots 312 allows the system 300 to adjust to changes in bone positioning caused by patient movement and healing. Simultaneously, the deformable bridge 316 can assist in maintaining constant compression, promoting healing large bones with significant mechanical loads.

The orthopedic staple system can be configured to be versatile in adapting to various surgical requirements and bone healing scenarios. The design configurations shown enable that the staple system can provide continuous, effective compression tailored to the specific dynamics of the surgical site and the healing process.

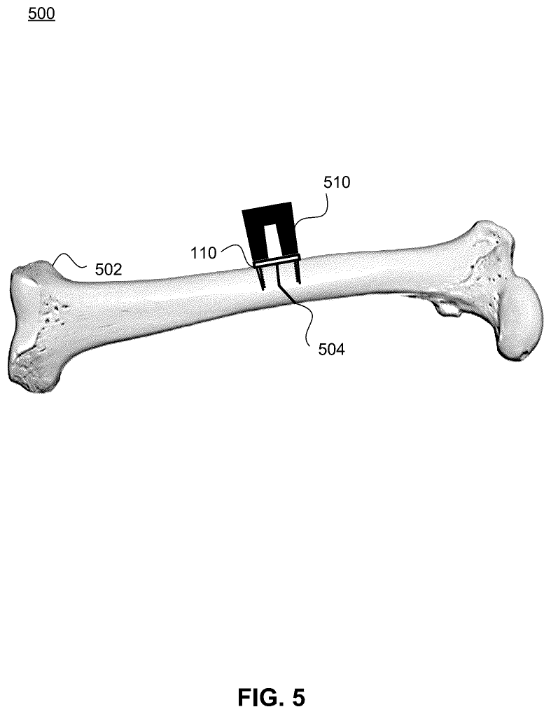

FIG. 5 depicts the surgical application 500 of orthopedic staple system 100 in a scenario involving minimally invasive techniques to treat a bone fracture. The illustration shows a surgeon using an insertion tool 410 to place the staple 110 accurately across the fracture site 504 on bone 502. The staple 110, prepared with a pre-compressed chevron-shaped bridge 116, can be aligned and positioned through a small incision that minimizes soft tissue disruption.

During the procedure, standard surgical tools such as driver 410 can be utilized to ensure the legs of the staple 110 are securely anchored into the bone 502 on either side of the fracture 504. This anchoring is critical as it provides the stability required for effective healing. The compact design of the staple 110 allows it to be inserted with minimal impact on the surrounding tissue, promoting a faster and safer recovery process.

As healing occurs, the chevron bridge 116 of the staple 110 actively contributes to the compression across the fracture site 504. This dynamic compression is provided to maintain constant, optimal pressure on the healing bone, accommodating slight movements and encouraging proper bone regeneration. The design of the staple 110 ensures that compression can be maintained and appropriately distributed across the site, enhancing the healing process without the need for further surgical intervention.

Post-operatively, the staple's placement and the progress of the bone healing can be monitored using standard imaging technology. This imaging can be performed to confirm correct placement of the staple 110 and assessing the ongoing alignment and stability of the bone 502. The ability to visually monitor the staple 110 and the fracture site 504 through imaging can ensure that any adjustments to the patient's care plan are based on the most accurate and current information available.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments but should be defined only in accordance with the following claims and their equivalents.

What is claimed is:

1. An orthopedic staple, comprising:
a bridge portion;
a first leg and a second leg extending from opposite ends of the bridge portion, wherein each leg is configured to penetrate and securely anchor into a bone structure,
wherein the bridge portion comprises a deformable configuration configured to exert a compressive force across a fracture site in the bone structure when the staple is implanted across the fracture site; and
wherein the bridge portion is further configured to enable dynamic adjustment of the compressive force in response to changes in the bone structure during a healing process.

2. The orthopedic staple of claim 1, further comprising a screw configured to interact with the bridge portion to apply compressive force across the fracture site.

3. The orthopedic staple of claim 1, further comprising:
a plurality of screws configured to interact with the bridge portion, each screw independently adjustable to apply a targeted compressive force across the fracture site.

4. The orthopedic staple of claim 2, wherein a head of the screw is configured to be seated in a variable angle pocket located in the bridge portion, the pocket configured to secure the screw head without allowing translational movement.

5. The orthopedic staple of claim 2, wherein a head of the screw is configured to be seated in a pocket within the bridge element, the pocket configured to permit translational movement of the screw head to accommodate compression and remodeling of the bone structure.

6. The orthopedic staple of claim 1, wherein the bridge portion is configured to provide active compression post-operatively.

7. The orthopedic staple of claim 6, wherein the active compression of the bridge portion is enabled by integration of elastically deformable polygons into the bridge portion.

8. The orthopedic staple of claim 6, wherein the active compression of the bridge portion is enabled by a deformable bridge path configured in an “S” shape.

9. The orthopedic staple of claim 1, further comprising one or more fixation screws configured as Active compression devices, enhancing compressive force applied during and after surgical placement.

10. An orthopedic staple for use in bone surgery, comprising:
a bridge portion;
a single leg extending from one end of the bridge portion, wherein the leg is configured to penetrate and securely anchor into bone structure;
a compression portion integrated into the bridge portion opposite the single leg, the compression portion configured to apply a targeted compressive force to the bone structure; and
the bridge portion including a mechanism to adjust the compressive force post-deployment to accommodate healing dynamics of the bone.

11. The orthopedic staple of claim 10, further comprising:
a single screw configured to interact with the bridge element to apply compressive force across the fracture site.

12. The orthopedic staple of claim 10, further comprising:
a plurality of screws configured to interact with the bridge element, each screw independently adjustable to apply a targeted compressive force across the fracture site.

13. The orthopedic staple of claim 11, wherein the bridge comprises a variable angle pocket, and a head of the screw is seated in the pocket, configured to secure the screw head without permitting translational movement.

14. The orthopedic staple of claim 11, wherein the bridge comprises a pocket, and a head of the screw is configured to seat in the pocket to permit translational movement of the head of the screw to accommodate compression and remodeling of the site.

15. The orthopedic staple of claim 10, wherein the bridge is configured to provide active compression post-operatively.

16. The orthopedic staple of claim 15, wherein:
the active compression of the bridge is enabled by the integration of elastically deformable polygons.

17. The orthopedic staple of claim 15, wherein:
the active compression of the bridge is accomplished by a deformable bridge path configured in an “S” shape.

18. The orthopedic staple of claim 10, further comprising one or more fixation screws configured as Active compression devices, enhancing compressive force applied during and after surgical placement.

19. An orthopedic staple system, comprising:
an orthopedic staple comprising a bridge portion and at least one leg extending from the bridge portion, the at least one leg configured to penetrate into a bone structure;
a compression screw configured to interact with the bridge element of the orthopedic staple to apply compressive force across a fracture site of the bone structure; and
a driver specifically adapted to engage with and manipulate the compression screw and the orthopedic staple during surgical implantation,
wherein the bridge element of the staple configured to dynamically adjust the compressive force exerted by the compression screw in response to physiological changes at the fracture site, facilitating healing conditions.

20. The orthopedic staple system of claim 19, wherein the head of the one or more screws are seated in a pocket located in the bridge element, wherein the pocket is selected from a variable angle pocket configured to secure the screw head without permitting translational movement or is a non-variable angle pocket that permits translational movement of the screw head to enable compression and remodeling of the fracture site.
